(19) United States
(12) Patent Application Publication
(10) Pub. No.: US 2016/0114044 A1
(43) Pub. Date: Apr. 28, 2016

(54) COMPOSITON BASED ON BIOCOMPATIBLE ANIONIC POLYMER FOR DRUG DELIVERY AND PREPARING METHOD THEREOF

(71) Applicants: RESEARCH & BUSINESS FOUNDATION SUNGKYUNKWAN UNIVERSITY, Suwon-si (KR); BIOLEADERS CORPORATION, Daejeon (KR)

(72) Inventors: Yong Taik LIM, Seongnam-si (KR); Seung-Pyo HONG, Daejeon (KR)

(73) Assignees: RESEARCH & BUSINESS FOUNDATION SUNGKYUNKWAN UNIVERSITY, Suwon-si (KR); BIOLEADERS CORPORATION, Daejeon (KR)

(21) Appl. No.: 14/836,779

(22) Filed: Aug. 26, 2015

(30) Foreign Application Priority Data
Aug. 27, 2014 (KR) 10-2014-0112655

(51) Int. Cl. A61K 47/32; A61K 31/4745; A61K 31/337

(52) U.S. Cl. CPC A61K 47/32; A61K 31/337; A61K 31/4745

(57) ABSTRACT

A composition for drug delivery based on a biocompatible anionic polymer and a producing method thereof are provided.

### CROSS-REFERENCE TO RELATED APPLICATION

This application claims the benefit of Korean Patent Application No. 10-2014-0112655 filed on Aug. 27, 2014 in the Korean Intellectual Property Office, the entire disclosure of which is incorporated herein by reference for all purposes.

### TECHNICAL FIELD

The embodiments described herein pertain to a composition for drug delivery based on a biocompatible anionic polymer and a producing method thereof.

### BACKGROUND

Cancers have continued to be one of the leading causes of death, accounting for more than 1.5 million new cases and 0.5 million deaths. Tumor is a complex and heterogeneous structure involving coevolution of vasculature, tumor-resistant immune cells, and extracellular matrix including fibroblasts which assist tumor cells escape therapeutic intervention. While chemotherapy has been used as a prominent anti-cancer modality, anti-cancer drugs are known to have high incidence of side-effects and disease recurrence. Paclitaxel is a potent chemotherapeutic agent used for treatment of various cancers such as breast cancer, non-small cell lung cancers, ovarian cancer, malignant brain tumors, and a variety of other solid tumors. The clinical efficacy is jeopardized because of the systemic side effects, insolubility in water and low bioavailability of paclitaxel. While various new approaches for effective and targeted delivery of paclitaxel are in progress, it has become a desirable candidate drug for combination with various other modalities, but has gained limited success in clinical trials.

Immune system is indispensable aspect of tumor microenvironment because tumor has the ability to evade the immune system by various mechanisms such as suppression of tumor reactive T cells by transforming growth factor-β (TGF-β) secretion or through regulatory T cells or by direct upregulation of death ligands such as Fas-L. Consequently, immunotherapeutic approaches for cancer such as dendritic cells and T cells based vaccine, cytokines, toll-like receptor (TLR) agonists, viral vaccine, peptide based vaccine, and DNA based vaccines have gained importance. Lately, the role of TLR stimulation has been emphasized for cancer treatment, and combination of TLR agonists with other treatment modalities such as T cell modulation, anti-CTLA4 therapy or CD4OL plasmid DNA for cancer treatment has been reported. While TLR agonists are being explored for anti-cancer treatment, on a contrary, cancer cells are known to express TLRs, and TLR agonists are also found to facilitate tumor proliferation, metastasis and inhibit apoptosis. Thus, selection of a suitable TLR agonist for cancer therapy is a critical step. TLR-7 agonist, imiquimod, also known as R837 has been approved by FDA for topical administration in cancer therapy and diseases such as genital warts, actinic keratoses, superficial basal cell carcinoma and lentigo maligna. Imiquimod is capable of imparting cytotoxic T cells with enhanced anti-tumor properties. Imiquimod is also known to induce systemic immunity in cryosurgery patients. In clinical trials, although it was well-tolerated with minimum side effects, an effective therapeutic response was not observed. Because of the dual nature of TLR agonists in cancer, monotherapy using standalone TLR agonists is leading to inadequate treatment and has great scope for improvisation.

Taking into consideration the limitations of chemotherapy and immunotherapy, chemo-immunotherapy has emerged as a new branch of cancer research with highly promising results. The efficacy of chemotherapeutic agent can be increased if the host immunity is also taken into consideration. It has been found that in phase III trial, using combination of chemotherapeutic agent 5-fluorouracil (5-FU) and adriamycin with TLR3 agonist polyadenylic-polyuridylic acid (poly A:U) leads to significantly prolonged patient survival as compared to chemotherapy alone. In a study, combination of paclitaxel and TLR4 agonist illustrated 40% reduction in tumor in mice as compared to paclitaxel alone. In another report, it was demonstrated that pre-conditioning with a chemotherapeutic agent complemented by adoptive T cell transfer, viral vaccine and an immunostimulatory TLR agonist is capable of getting rid of melanoma tumor completely. These results suggest that if the tumor surveillance is broken, then even low concentrations of anti-cancer drug is expected to be effective with reduced side-effects. However, the challenge is to determine an appropriate combination of anticancer drug and TLR agonist, which can completely eliminate the tumor without any reappearance of the disease. Another consideration is that anti-cancer drug should have a minimum adverse effect on the immune cells while it should be able to kill tumor cells at the same concentrations of treatment. Also, the TLR agonist selected should not only be able to counter the immuno-suppressive environment within the tumor but should also be capable of triggering release of cytokines by immune cells, which is sufficient for development of an anti-tumor milieu.

Since most of conventional commercialized anticancer drugs and immune-active substances have a non-hydrophilic property, there is a problem to use various organic solvents and excipients are used in order to prepare formulations of the drugs and the substances. The problem is that the drugs and the substances can be administered only as liquid injections due to a severe adverse effect resulting from their toxicity. In addition, a process of preparing liposome or emulsion used to disperse a water-insoluble substance in an aqueous solution is cumbersome since it needs special preparation facilities. Further, since the period of time for formation of the emulsion is extremely short, the morphology of the emulsion remains for only a few minutes immediately after mixing, and phase separation occurs soon (refer to Korean Patent Application Publication Nos. 10-2003-0003320 and 10-2004-0066300).

### SUMMARY

In view of the foregoing, embodiments provide a composition for drug delivery based on a biocompatible anionic polymer and a producing method thereof.

However, the problems sought to be solved by the present disclosure are not limited to the above description, and other problems can be clearly understood by those skilled in the art from the following description.

The first aspect of the present disclosure provides a composition for drug delivery, which includes a biocompatible anionic polymer; an agonist of an immunostimulant; and an active pharmaceutical ingredient.

The second aspect of the present disclosure provides a method of producing a composition for drug delivery of the first aspect, comprising: (a) adding a biocompatible anionic polymer into an agonist of the immunostimulant dispersed in an organic solvent under ultrasonication to prepare a dispersion of the biocompatible anionic polymer-the agonist of the immunostimulant, and then, lyophilizing the dispersion to remove the organic solvent to obtain a powder of the agonist of the immunostimulant dispersed in the biocompatible anionic polymer; (b) adding a biocompatible anionic polymer into an active pharmaceutical ingredient dispersed in an organic solvent under ultrasonication to prepare a dispersion of the biocompatible anionic polymer—the active pharmaceutical ingredient, and then, lyophilizing the dispersion to remove the organic solvent to obtain a powder of the active pharmaceutical ingredient dispersed in the biocompatible anionic polymer; and (c) redispersing the powders obtained from the steps (a) and (b) in distilled water.

The third aspect of the present disclosure provides a composition for anticancer therapy, including the composition for drug delivery according to the first aspect as an active ingredient.

In accordance with the embodiments, preparation of a γ-PGA-based formulation is a convenient, single step synthesis process (mixing γ-PGA and a drug in a co-solvent followed by freeze-drying) and is amenable to scale up.

In accordance with the embodiments, since the preparation of a γ-PGA-based formulation does not involve synthesis steps such as washing or centrifugation (as in other delivery systems such as nanoparticles, liposomes and polymer matrices), there is no loss of drug, thereby, providing 100% encapsulation efficiency of drug.

### BRIEF DESCRIPTION OF THE DRAWINGS

FIG. 1 is a schematic view showing an in-vivo tumor killing mechanism of a composition for drug delivery in accordance with an Example of the present disclosure.

FIG. 2 shows scanning electron microscope (SEM) images of compositions for drug delivery (γ-PGA/Ptx, γ-PGA/Imq and γ-PGA/Ptx/Imq) in accordance with an Example of the present disclosure.

FIG. 3 shows a table for a particle size and a polydispersity index of the compositions for drug delivery (γ-PGA/Ptx, γ-PGA/Imq and γ-PGA/Ptx/Imq) in accordance with an Example of the present disclosure.

FIG. 4 shows the dispersion status of pacrlitaxel, imiquimod or a mixture thereof, which has been dispersed in y-PGA and distilled water, respectively, according to lapse of time.

FIG. 5 shows graphs for cell viability for 24 hours and 48 hours after the compositon for drug delivery in accordance with an Example of the present disclosure is treated for (A) B-16 melanoma cells, (B) HeLa breast cancer cells, and (C) A549 lung cancer cells, respectively.

FIG. 6 shows graphs for cell viability for 24 hours and 48 hours after the composition for drug delivery in accordance with an Example of the present disclosure is treated for (A) RAW264.7 immune cells and (B) BMDCs immune cells, respectively.

FIG. 7 shows graphs for BMDCs activity by the composition for drug delivery in accordance with an Example of the present disclosure, and provides results of measurement of cytokine secretion of (A) IL-12, (B) TNF-α, (C) IL-6, and (D) IL-1β.

FIG. 8 shows graphs for maturation of BMDCs by the composition for drug delivery in accordance with an Example of the present disclosure, and provides results of measurement of a cell surface expression extent of (A) CD80, (B) MHClI, and (C) MHCl.

FIG. 9 shows graphs for a tumor volume and mouse survival in accordance with an Experimental Example of the present disclosure, in which the tumor volume was measured until 23 days after injection of samples.

FIG. 10 shows graphs for a tumor volume and mouse survival after a concentration of paclitaxel in γ-PGA/Ptx increased to 200 μg in accordance with an Experimental Example of the present disclosure.

FIG. 11 shows proliferation of immune cells in tumor draining lymph nodes on a 16^{th} day after administration of the composition for drug delivery in accordance with an Experimental Example of the present disclosure.

FIG. 12 shows maturation of CD11c⁺ dendritic cells through an expression volume of (A) CD40, (B) CD80, (C) CD86, and (D) MHClI in accordance with an Experimental Example of the present disclosure.

### DETAILED DESCRIPTION

Hereinafter, embodiments will be described in detail with reference to the accompanying drawings so that inventive concept may be readily implemented by those skilled in the art. However, it is to be noted that the present disclosure is not limited to the embodiments but can be realized in various other ways. In the drawings, certain parts not directly relevant to the description are omitted to enhance the clarity of the drawings, and like reference numerals denote like parts throughout the whole document.

Throughout the whole document, the terms “connected to” or “coupled to” are used to designate a connection or coupling of one element to another element and include both a case where an element is “directly connected or coupled to” another element and a case where an element is “electronically connected or coupled to” another element via still another element.

Throughout the whole document, the term “on” that is used to designate a position of one element with respect to another element includes both a case that the one element is adjacent to the another element and a case that any other element exists between these two elements.

Throughout the whole document, the term “comprises or includes” and/or “comprising or including” used in the document means that one or more other components, steps, operations, and/or the existence or addition of elements are not excluded in addition to the described components, steps, operations and/or elements.

Throughout the terms “about or approximately” or “substantially” are intended to have meanings close to numerical values or ranges specified with an allowable error and intended to prevent accurate or absolute numerical values disclosed for understanding of the present invention from being illegally or unfairly used by any unconscionable third party.

Throughout the whole document, the term “step of” does not mean “step for.”

Throughout the whole document, the term “combination of” included in Markush type description means mixture or combination of one or more components, steps, operations and/or elements selected from a group consisting of components, steps, operation and/or elements described in Markush type and thereby means that the disclosure includes one or more components, steps, operations and/or elements selected from the Markush group.

Throughout the whole document, the description “A and/or B” means “A or B, or A and B.”

Throughout the whole document, the term “object” means a target, which needs treatment of a disease, and more specifically, mammals such as a human being or a non-human primate, a mouse, a rat, a dog, a cat, a horse, and a cow, etc.

Throughout the whole document, the term “pharmaceutically effective dose” means an amount sufficient to prevent or treat a disease, preferably, achieve a prevention effect, and a range of a “pharmaceutically effective dose” can be variously adjusted depending on a weight, age, gender, a health condition, diet, administration time, an administration method, an excretion rate, disease severity and others of a patient who is a target for administration.

Embodiments of the present disclosure have been described in detail, but the present disclosure may not be limited to the embodiments.

The first aspect of the present disclosure provides a composition for drug delivery, which includes a biocompatible anionic polymer; an agonist of an immunostimulant; and an active pharmaceutical ingredient.

Effective delivery of a water-insoluble therapeutic agent is one of the major hurdles in their clinical application. Various approaches such as solid dispersions, micro-suspensions and nano-suspensions by wet milling process, melt extrusion or using stabilizers have been explored. The selected anti-cancer drug, paclitaxel, and an immuno-stimulating agent, imiquimod, are naturally water-insoluble in nature. Therefore, the primary aim of an embodiment of the present disclosure is effective administration of the two kinds of drugs, for which water soluble polymer γ-PGA is used, and the drugs are allowed to form a microdispersion using a co-solvent.

In an embodiment of the present disclosure, a γ-PGA-based formulation is particularly beneficial in combination therapy as combinations of various drugs can be readily dispersed in a desired ratio without causing any loss or disturbing the relative dose of individual drugs. γ-PGA has free carboxyl groups, which are expected to have noncovalent interactions such as hydrogen bonding with amine groups present in drug molecules leading to effective dispersion of drug crystals. The stability of the dispersion can also be attributed to the viscosity of the γ-PGA solutions, which prevents precipitation of dispersed micro-crystals. The dispersion is stable without any phase separation up to 6 months.

Especially, one of the most important aims of all anti-cancer immunotherapy is the induction of a potent tumor specific immune response by enabling immune cells to recognize and kill the tumor cells. While cell-based therapy involving dendritic cells, T cell-based or serial killer cell-based therapy has been gaining importance, and are undergoing clinical evaluation, these procedures involve multi-step patient procedures. Administration of immune-stimulating agents to produce a robust immune response in-situ can be a convenient approach for anti-cancer immunotherapy. Combination of an immuno-enhancing agent with a low amount of an anti-cancer drug in accordance with an embodiment of the present disclosure can facilitate production of a tumor specific antigen.

In an embodiment of the present disclosure, the anionic polymer, the agonist of the immunostimulant and the active pharmaceutical ingredient may be included in a weight ratio of about 1: about 0.01 to about 300: about 0.01 to about 300, but not be limited thereto.

In an embodiment of the present disclosure, the biocompatible anionic polymer may include a member selected from the group consisting of carboxyl group, hydroxyl group, sulfonic group, sulfate group and combinations thereof, but not be limited thereto.

In an embodiment of the present disclosure, the biocomplatible anionic polymer may include a member selected from the group consisting of a poly-gamma-glutamic acid, a hyaluronic acid, cellulose, a polyacrylic acid, a polyamino acid, a polysaccharide, derivatives thereof, and combinations thereof, but not be limited thereto.

In an embodiment of the present disclosure, the immunostimulant may include a toll-like receptor (TLR), a NOD-like receptor, or cytokine, but not be limited thereto. The toll-like receptor is mostly expressed in immune cells to perform a key role in immune activity, and known to increase maturation of a dendritic cell through stimulus of the active pharmaceutical ingredient. The toll-like receptor may include a member selected from the group consisting of, for example, TLR-1, TLR-2, TLR-3, TLR-4, TLR-5, TLR-6, TLR-7, TLR-8 and TLR-9, but not be limited thereto. The NOD-like receptor, as an intracellular sensor of pathogen-associated molecular patterns (PAMPs) entering into cells through phagocytosis or pores and damage-associated molecular pattern molecules (DAMPS) associated with cell stress, is a part of a pattern recognition receptor and plays an important role in an innate immune response. The NOD-like receptor may include, for example, NLRA, NLRB, NLRC or NLRP, but not be limited thereto. The cytokine is a generic name of proteins secreted by immune cells, and known to induce proliferation of macrophages or promote its differentiation. The cytokine may include, for example , IL-1α, IL-113, IL-1, IL-2, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IL-24, IL-25, GM-CSF, G-CSF, M-CSF, TNF-α, TNF-β, IFNα, or IFNβ, but not be limited thereto.

In an embodiment of the present disclosure, the agonist of the immunostimulant may include poly (I:CU), CpG, imiquimod, resiquimod, dSLIM, MPLA, flagellin, a plasmid DNA double-strand DNA, a single-strand DNA, a saponine, and an interleukin cytokine, but not be limited thereto.

In an embodiment of the present disclosure, the active pharmaceutical ingredient may include an anticancer agnet, but not be limited thereto.

In an embodiment of the present disclosure, the anticancer drug may include a member selected from the groups consisting of paclitaxel, docetaxel, doxorubicin, adriamycin, cis-platin, mitomycin-C, daunomycin, 5-fluorouracil, griseofulvin, digoxin, dipyridamol, spironolactone, cyclosporine, amphotericin B, etoposide, 6-mercaptopurine, dexamethasone, perphenazine, 20-S-camptothecin, 9-nitro-camptothecin, 9-amino-camptothecin, 10,11-methylenedioxy-camptothecin, taxol, taxol-A, mitotane, methotrexate, lomustine, interferon, rouracil, and etoposide, but not be limited thereto.

In an embodiment of the present disclosure, the biocompatible anionic polymer, the agonist of the immunostimulant, and the active pharmaceutical ingredient may be linked to one another via intermolecular non-covalent bond, but not be limited thereto.

In an embodiment of the present disclosure, the biocompatible anionic polymer and the agonist of the immunostimulant in the agonist of the immunostimulant dispersed in the biocompatible anionic polymer may be included in a weight ratio of about 1: about 0.01 to about 300;

The biocompatible anionic polymer and the active pharmaceutical ingredient in the active pharmaceutical ingredient dispersed in the biocompatible anionic polymer may be included in a weight ratio of about 1: about 0.01 to about 300; and

The agonist of the immunostimulant dispersed in the biocompatible anionic polymer and the active pharmaceutical ingredient dispersed in the biocompatible anionic polymer may be included in a weight ratio of about 1: about 1 to about 5, but the present disclosure may not be limited thereto.

The second aspect of the present disclosure provides a method of producing a composition for drug delivery of the first aspect, comprising: (a) adding a biocompatible anionic polymer into an agonist of the immunostimulant dispersed in an organic solvent under ultrasonication to prepare a dispersion of the biocompatible anionic polymer-the agonist of the immunostimulant, and then, lyophilizing the dispersion to remove the organic solvent so as to obtain a powder of the agonist of the immunostimulant dispersed in the biocompatible anionic polymer; (b) adding a biocompatible anionic polymer into an active pharmaceutical ingredient dispersed in an organic solvent under ultrasonication to prepare a dispersion of the biocompatible anionic polymer—the active pharmaceutical ingredient, and then, lyophilizing the dispersion to remove the organic solvent so as to obtain a powder of the active pharmaceutical ingredient dispersed in the biocompatible anionic polymer; and (c) redispersing the powders obtained from the steps (a) and (b) in distilled water.

In an embodiment of the present disclosure, the biocompatible anionic polymer may include a member selected from the group consisting of a carboxyl group, a hydroxyl group, a sulfonic group, a sulfate group, and combinations thereof, but not be limited thereto.

In an embodiment of the present disclosure, for the organic solvent, any organic solvent may be used without limitation if it can dissolve the agonist of the immunostimulant and the active pharmaceutical ingredient.

In an embodiment of the present disclosure, the biocompatible anionic polymer may include a member selected from the group consisting of a poly-gamma-glutamic acid, a hyaluronic acid, cellulose, a polyacrylic acid, a polyamino acid, a polysaccharide, derivatives thereof, and combinations thereof, but not be limited thereto.

In an embodiment of the present disclosure, the immunostimulant may include a toll-like receptor, an NOD-like receptor, or a cytokine, but not be limited thereto. The toll-like receptor is mostly expressed in immune cells to perform a key role in immune activity, and known to increase maturation of a dendritic cell through stimulus of the active pharmaceutical ingredient. The toll-like receptor may include a member selected from the group consisting of TLR-1, TLR-2, TLR-3, TLR-4, TLR-5, TLR-6, TLR-7, TLR-8 and TLR-9, but not be limited thereto. The NOD-like receptor, as an intracellular sensor of pathogen-associated molecular patterns (PAMPs) entering into cells through phagocytosis or pores and damage-associated molecular pattern molecules (DAMPS) associated with cell stress, is a part of a pattern recognition receptor and plays an important role in an innate immune response. The NOD-like receptor may include, for example, NLRA, NLRB, NLRC or NLRP, but not be limited thereto. The cytokine is a generic name of proteins secreted by immune cells, and known to induce proliferation of macrophages or promote its differentiation. The cytokine may include, for example, IL-1α, IL-113, IL-1, IL-2, IL-3, IL-4, IL-5, IL-6, IL-7, IL-8, IL-9, IL-10, IL-11, IL-12, IL-13, IL-15, IL-16, IL-17, IL-18, IL-19, IL-20, IL-21, IL-22, IL-23, IL-24, IL-25, GM-CSF, G-CSF, M-CSF, TNF-α, TNF-β, IFNα, or IFNβ, but not be limited thereto.

In an embodiment of the present disclosure, the agonist of the immunostimulant may include poly(I:CU), CpG, imiquimod , resiquimod, dSLIM, MPLA, flagellin, a plasmid DNA double-strand DNA, a single-strand DNA, a saponin, or an interleukin cytokine, but not be limited thereto.

In an embodiment of the present disclosure, the active pharmaceutical ingredient may include an anticancer agent, but not be limited thereto.

In an embodiment of the present disclosure, the anticancer agent may include a member selected from the group consisting of paclitaxel, docetaxel, doxorubicin, adriamycin, cis-platin, mitomycin-C, daunomycin, 5-fluorouracil, griseofulvin, digoxin, dipyridamol, spironolactone, cyclosporine, amphotericin B , etoposide, 6-mercaptopurine, dexamethasone, perphenazine, 20-S-camptothecin, 9-nitro-camptothecin, 9-amino-camptothecin, 10,11-methylenedioxy-camptothecin, taxol, taxol-A, mitotane, methotrexate, lomustine, interferon, rouracil, and etoposide, but not be limited thereto.

In an embodiment of the present disclosure, the agonist of the immunostimulant dispersed in the biocompatible anionic polymer and the active pharmaceutical ingredient may be linked to each other via an intermolecular non-covalent bond, but not be limited thereto.

The third aspect of the present disclosure provides a composition for anticancer therapy, including the composition for drug delivery according to the first aspect as an active ingredient.

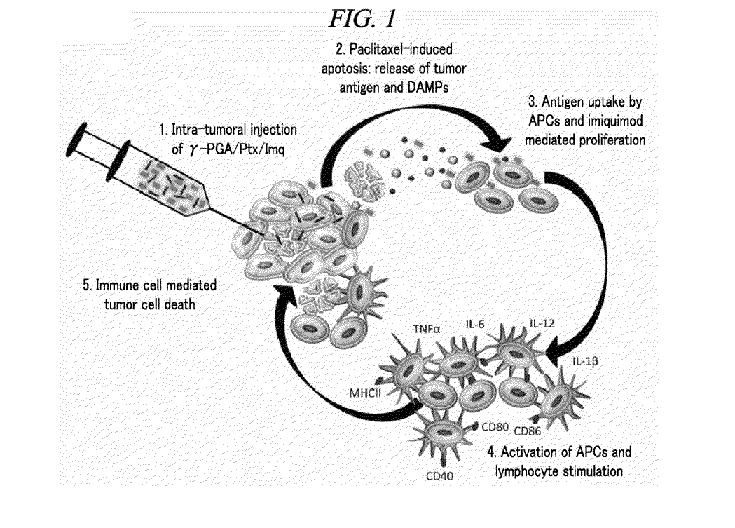

According to an embodiment of the present disclosure, since the agonist of the immunostimulant and the active pharmaceutical ingredient are dispersed in water with the help of poly(γ-glutamic acid) using a co-solvent system leading to formation of micro-dispersions of drugs, and can significantly inhibit growth of an inter-tumor, it can be applied to various drug delivery systems (FIG. 1).

In an embodiment of the present disclosure, the composition for anticancer therapy may include a pharmaceutically acceptable carrier, but not be limited thereto. The pharmaceutically acceptable carrier may include physiological saline, polyethylene glycol, ethanol, vegetable oil, isopropyl myristate and others, but not be limited thereto.

In an embodiment of the present disclosure, the composition for anticancer therapy may be administered in a pharmaceutically effective amount to an object, but not be limited thereto. The pharmaceutically effective amount may be variously adjusted depending on a weight, age, gender, a health condition, diet, administration time, an administration method, an excretion rate, and disease severity of a patient who is a target for administration.

In an embodiment of the present disclosure, an administration amount of the composition for anticancer therapy may be properly selected by one of ordinary skill in the art of the present disclosure, though it varies depending on condition and weight of a patient, disease severity, drug forms, administration routes, and time periods. However, preferably, the composition may be administered in about 0.001 mg/kg to about 100 mg/kg per day, and for example, about 0.01 mg/kg to about 50 mg/kg, but not be limited thereto. The composition may be administered once or several times a day. In an embodiment of the present disclosure, the composition for anticancer therapy may be present in an amount of about 0.0001 wt % to about 50 wt %, for example, about 0.001 wt % to about 10 wt % based on a total composition weight.

In an embodiment of the present disclosure, the composition for anticancer therapy may be administered to mammals including a human being through various routes. The route of administration is not limited, and for example, may be oral administration, rectal administration, intraperitoneal administration, intrapulmonary administration, intranasal administration, intravenous administration, intramuscular administration, subcutaneous administration, intrauterine epidural administration, or intracerbroventricular administration.

In an embodiment of the present disclosure, cancers to be treated by the composition for anticancer therapy are not limited and may include, for example, but not be limited to, a breast cancer, an ovarian cancer, a cervical cancer, an endometrial cancer, a lung cancer, a lung adenocarcinoma, a cholangiocarcinoma, a colorectal cancer, melanoma, neuroblastoma, or a pancreatic cancer.

Descriptions of the third aspect of the present disclosure, which overlap with those of the first and second aspects, have been omitted; however, the descriptions of the first and second aspects can be identically applied to the third aspect, though they have been omitted.

Hereinafter, the present disclosure is described more in detail by using Examples, but the Examples are merely illustrative to facilitate the understanding of the present disclosure, and the present disclosure is not limited to the Examples.

### EXAMPLES

In this Example, poly(γ-glutamic acid) (γ-PGA) based combination of low dose of anti-cancer drug (paclitaxel) with an immunestimulatory agent (imiquimod) was tested for a synergetic effect against solid tumor. Paclitaxel treatment was speculated to cause tumor cell death, which should lead to production of tumor specific antigens as well as danger signals also known as damage associated molecular patterns (DAMPS). Imiquimod was used as an adjuvant and was expected to induce activation and maturation of immune cells for eliciting an anti-tumor immune response. Since both paclitaxel and imiquimod are water-insoluble drugs, γ-PGA, which is a water soluble bioderived anionic polymer, was used to form a stable aqueous micro-dispersion of the two drugs. The memory response against tumor was also assessed in order to test the longevity and clinical applicability of the formulation.

For analyzing the statistical difference between two groups unpaired t-tests with Welch's correction was used. For analyzing the statistical difference between more than 2 groups, two-way ANOVA with Bonferroni post-tests was used. For analyzing statistical significance in survival data, a log-rank (Mantele-Cox) test was used, and a p value<0.05 was considered statistically significant. All values are expressed as mean±standard deviation. GraphPad Prism software was used for all statistical analysis.

### Example 1

### Preparation of Polymer-Drug Micro-Dispersion

For preparation of γ-PGA/paclitaxel micro-dispersion (γ-PGA/Ptx), 10 mg of paclitaxel (ChemieTek, Indianapolis, USA) in 500 μL DMSO was added to 2 mL of 2.5% (w/v) y-PGA (50 kDa) (BioLeaders Corporation, Daejeon, South Korea) while sonicating using a probe sonicator for 2 min. For preparation of γ-PGA imiquimod micro-dispersion (γ-PGA/Imq), 10 mg of imiquimod (TCl, Tokyo, Japan) was dissolved in 2 mL DMSO by heating at 60° C. This solution was added to 2 mL of 2.5% γ-PGA (50 kDa) while sonicating using a probe sonicator for 2 min. The above solutions were lyophilized for removal of organic solvents. The lyophilized powder was redispersed in distilled water to obtain a stable dispersion. For preparation of y-PGA/paclitaxel/imiquimod micro-dispersion (γ-PGA/Ptx/Imq) for combination therapy, the redispersed solutions made above were mixed in a weight ratio of 1:5 (γ-PGA/Ptx: γ-PGA/Imq).

### Experimental Example 1

### Characterization of Micro-Dispersion

The particle size of the redispersed solutions of γ-PGA/Ptx and γ-PGA/Imq was measured using Electrophoretic Light Scattering-ELS Z (Otsuka Electronics, Osaka, Japan). The stability of the dispersion was assessed by observing the samples for aggregation and phase separation after storing the sample at 4° C. for 1 week after dispersion in distilled water. The morphology of theses micro-dispersions were observed through FE-SEM (JSM-7000F, JEOL Ltd. Japan) and FE-TEM (JEM-2100F HR, JEOL Ltd. Japan). For the SEM sample preparation, the lyophilized and redispersed suspension was deposited on a silicon wafer plate and vacuum-dried to remove water, followed by platinum coating using a Technics Hummer II sputter coater at 30 mA for 90 s. For TEM sample preparation, a drop (2 μL) of redispersed suspension was placed onto a formvar/carbon coated copper grid, followed by vacuum-drying to evaporate water.

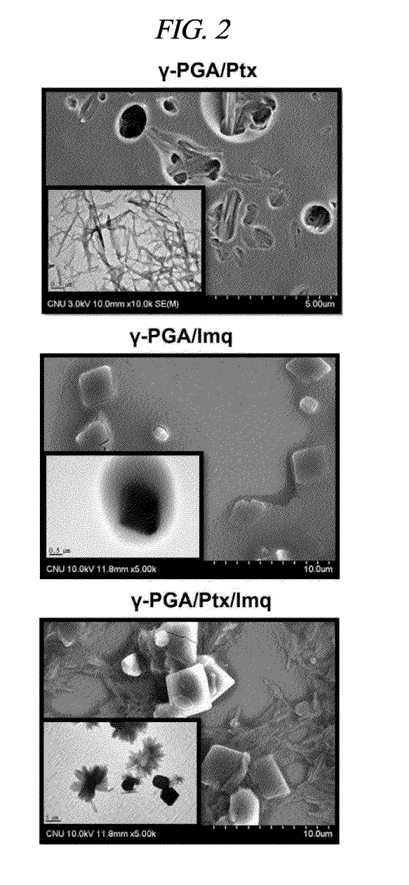

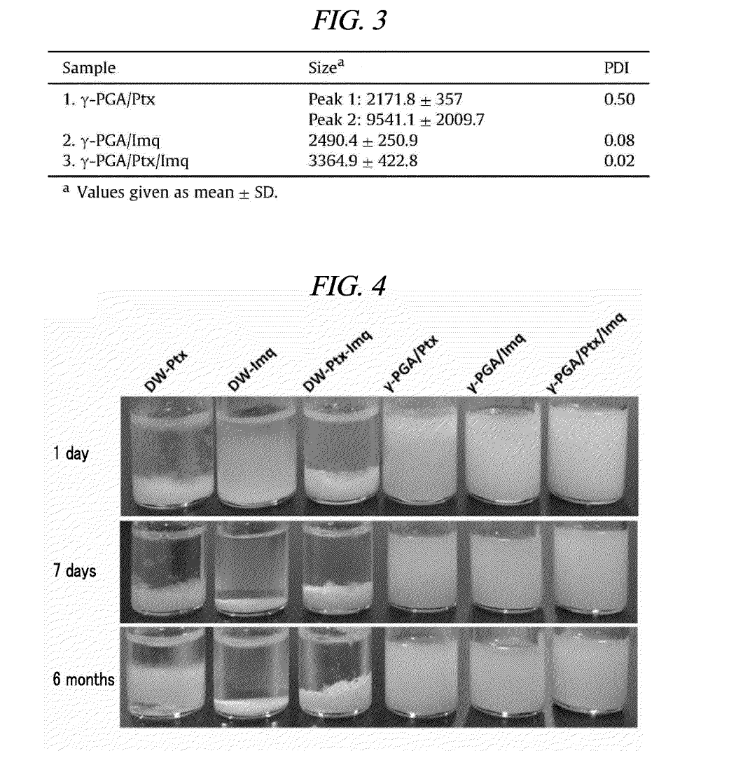

As shown in FIG. 2, the electron micrographs of the dried micro-dispersions showed presence of elongated paclitaxel and cubic imiquimod crystals embedded in a polymeric matrix of γ-PGA. From the TEM images, well dispersed crystals of individual drugs were clearly demonstrated. FIG. 3 shows a table for a particle size and a polydispersity index of the compositions for drug delivery (γ-PGA/Ptx, γ-PGA/Imq and γ-PGA/Ptx/Imq) and provides results of analysis of a size and size distribution of an aqueous dispersion of the lyophilized microdispersion. Distinct monodisperse peaks for γ-PGA/Imq and γ-PGA/Ptx/Imq were observed, indicating uniform size distribution (FIG. 3). In case of γ-PGA/Ptx, two peaks were observed, which might be due to a high aspect ratio of the paclitaxel crystal. FIG. 4 shows the dispersion state of pacrlitaxel, imiquimod or a mixture thereof, which has been dispersed in γ-PGA and distilled water, respectively, according to lapse of time. As shown in FIG. 4, it was demonstrated that all the suspensions were well dispersed in a γ-PGA matrix for 7 days and could be stably redispersed for longer times (more than 6 months) as well (FIG. 4). However, paclitaxel, imiquimod and combination of both the drugs, when they were dispersed in water without γ-PGA, led to aggregation and precipitation due to their hydrophobicity.

### Experimental Example 2

### In-vitro Viability Assay

Tumor cells (A549 human lung adenocarcinoma epithelial cell line, B-16 murine melanoma cell line, HeLa human cervical cancer cell line) and immune cells (bone marrow derived dendritic cells-BMDCs and RAW264.7 macrophages) were tested for in-vitro viability after treatment with various concentrations of γ-PGA/Ptx, γ-PGA/Imq and γ-PGA/Ptx/Imq. All the tumor cell lines and RAW264.7 were purchased from ATCC. BMDCs were isolated from mouse bone marrow as reported previously. MTS assay for analysis of mitochondrial activity was used as a measure of cell viability. 1×10⁴ cells in 100 μL media were seeded per well in a 96 well plate (Corning Costar, Cambridge, Mass., USA). 100 μl of sample dispersion was added to cell culture media and incubated for 24 h and 48 h at 37° C. and 5% CO₂. After the incubation, 20 μL of MTS solution-Cell Titer 96 Aqueous One Solution kit (Promega, Madison, Wis., USA) was added to each well and incubated for 2 h. The absorbance of the samples was measured at 490 nm (VersaMax, Molecular Devices, Sunnyvale, Calif., USA) and normalized with respect to absorbance of untreated cells.

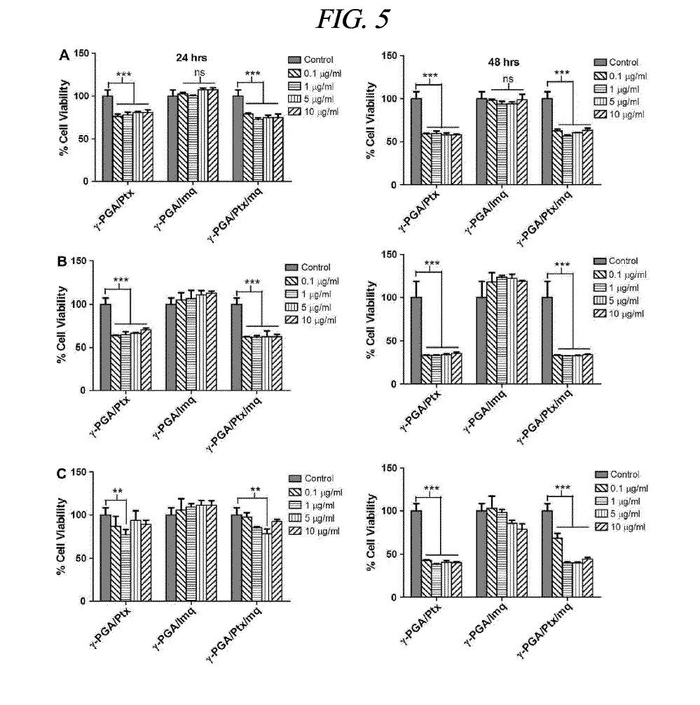

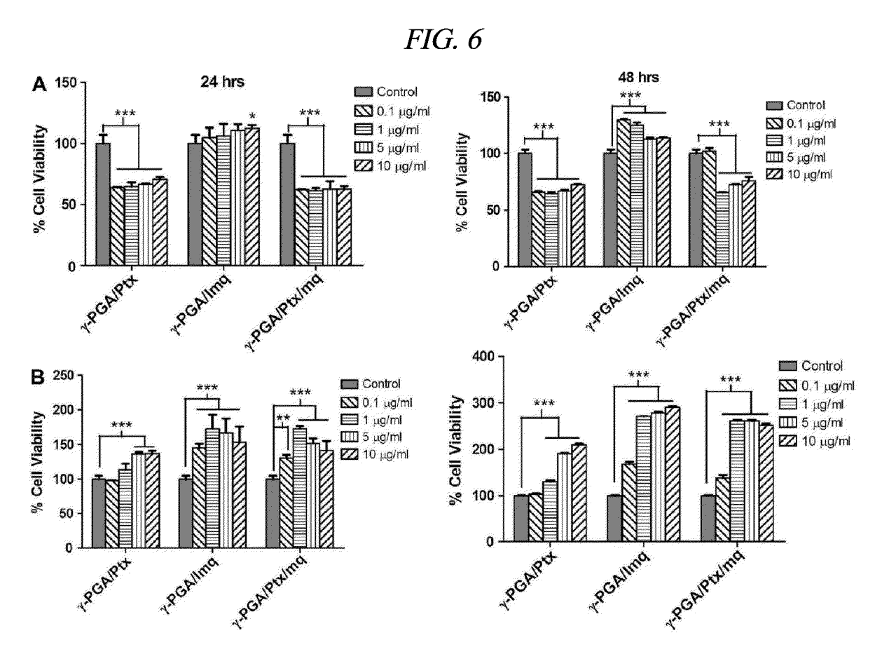

The three different tumor cell lines were tested for viability in the presence of different concentrations (Ptx—0.1, 1, 5 and 10 μg/mL) of paclitaxel in γ-PGA/Ptx and same concentrations (Imq—0.1, 1, 5 and 10 μg/mL) of imiquimod in γ-PGA/Imq after 24 h and 48 h. For testing the combination of γ-PGA/Ptx/Imq, the concentration of imiquimod was 0.1 μg/mL, 1 μg/mL, 5 μg/mL and 10 μg/mL, while the concentration of paclitaxel was ⅕th of the concentration of imiquimod for each sample. FIG. 5 shows graphs for cell viability for 24 h and 48 h after the composition for drug delivery is treated for (A) B-16 melanoma cells, (B) HeLa breast cancer cells, and (C) A549 lung cancer cells, and for B-16 melanoma, A549 lung cancer and HeLa cervical cancer cells, both γ-PGA/Ptx and γ-PGA/Ptx/Imq had a significant cytotoxic effect after 48 h. Each of the data of γ-PGA had a value of **p<0.01, ***p<0.001 by two-way ANOVA, with the Bonferroni post-test (FIG. 5). In all the three tumor cell lines, γ-PGA/Imq did not have any significant cytotoxic effect indicative of lack of direct antitumor activity of imiquimod. Paclitaxel is a mitotic inhibitor, which stabilizes the microtubule polymer and protects it from disassembly. Because the mechanism of action of paclitaxel is nonspecific, it also damages normal cells and is thus known to be associated with many side-effects. FIG. 6 shows graphs for cell viability for 24 h and 48 h after the composition for drug delivery is treated for (A) RAW264.7 immune cells and (B) BMDCs immune cells, respectively, and cytotoxic effects of the composition were observed on RAW264.7 macrophages while γ-PGA/Imq was not cytotoxic (FIG. 6A). Each of the data of FIG. 6 had a value of *p<0.05, **p<0.01, ***p<0.001 by two-way ANOVA, with the Bonferroni post-test.

Interestingly, all the three samples had a proliferation enhancing effect on BMDCs. γ-PGA/Ptx had a dose dependent increase in proliferation of dendritic cells, indicating dendritic cells are not only resistant to cyto-toxicity at these concentrations of paclitaxel, but are also capable of enhanced proliferation. Similar results were also observed previously and are speculated to be due to the TLR4 stimulation of dendritic cells by paclitaxel. Also, γ-PGA/Imq and γ-PGA/Ptx/Imq had a remarkable increase (>250%) in cell viability after 48 h (FIG. 6B). This enhanced BMDC number in the presence of both paclitaxel and imiquimod is expected to induce massive proliferation of dendritic cells within the immunosuppressed microenvironment of a tumor.

### Experimental Example 3

### In-vitro BMDC Activation and Maturation

1×10⁶ BMDCs in 1 mL of RPMI media supplemented with 10% FBS and 1% antibiotic-antimycotic solution were seeded per well in a 6 well plate (Corning Costar, Cambridge, Mass., USA). 1 mL of a sample was treated, and cells were incubated for 24 h at 37° C. and 5% CO₂. The plate was centrifuged at 1500 rpm for 5 min, and the supernatant was collected and analyzed for various cytokines (IL-1β, IL-12, IL-6 and TNF-α) using ELISA kit (BD Biosciences). For analysis of upregulation of a maturation marker after incubating with samples for 24 h, the cells were harvested and fixed using 4% paraformaldehyde. The cells were treated with a fluorescence labeled marker antibody FITC-CD80 (BD PharMingen, San Diego, Calif., USA), PE-MHCl (eBioscience, San Diego, Calif., USA) and PE-MHClI (eBioscience, San Diego, Calif., USA) by incubating at 4° C. for 30 min followed by 2 times washing with PBS. 10⁴cells were then analyzed for fluorescence using MACS (Miltenyl Biotec). Data was further analyzed using a MACSQuant Analyzer (Miltenyi Biotec, Germany).

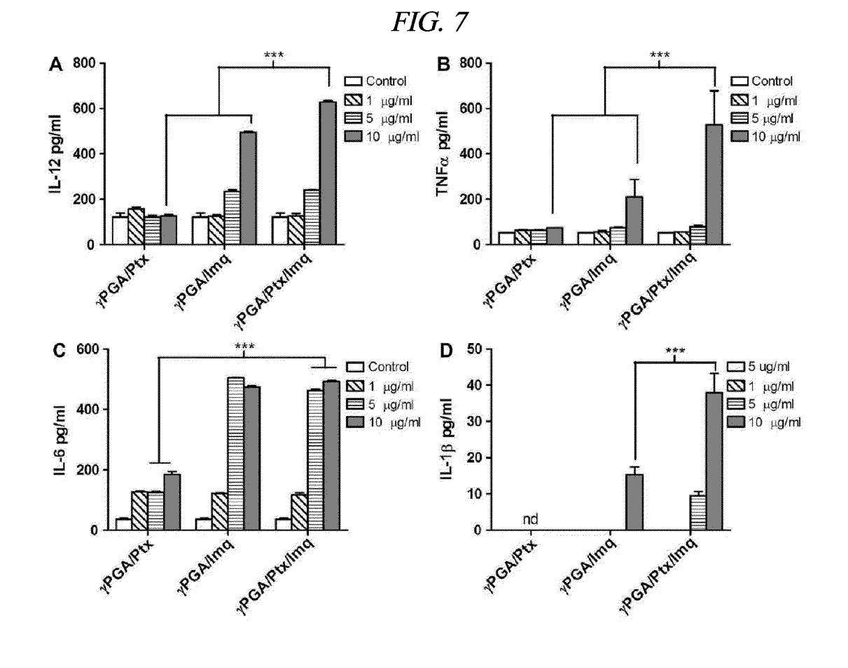

As shown in FIG. 7, the BMDCs maturation and activation were studied by analyzing the secretion levels of pro-inflammatory cytokines IL-6, IL-1β, TNF-α and Th1 cytokine (IL-12). Dose dependent increase in IL-12 secretion for immunostimulatory samples, γ-PGA/Imq and γ-PGA/Ptx/Imq were observed (FIG. 7A). IL-12 is known to facilitate differentiation of naïve T cells to Th1 cells. The secretion level of TNF-α, which is known to induce apoptotic cell death and inhibit tumorigenesis, was drastically enhanced in the presence of combination γ-PGA/Ptx/Imq (FIG. 7B). The secretion of pro-inflammatory cytokines IL-6 and IL-1β was also considerably enhanced in the presence of immunostimulatory samples γ-PGA/Imq and γ-PGA/Ptx/Imq (FIG. 7C and FIG. 7D). Each of the data of FIG. 7 had a value of **p<0.01, ***p<0.001 by two-way ANOVA, with the Bonferroni post-test.

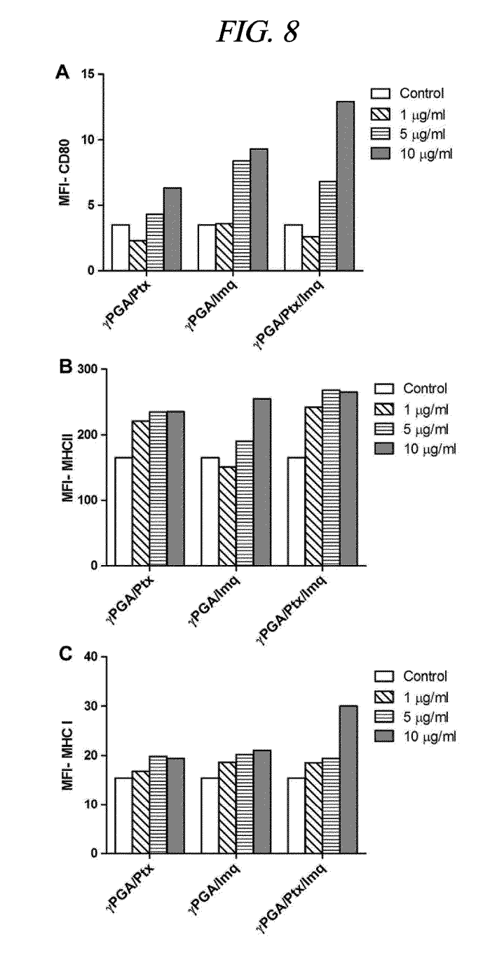

In addition, activation markers CD80, MHC II and MHC I were upregulated in the presence of all three types of γ-PGA microdispersions as compared to control. FIG. 8 shows graphs for maturation of BMDCs by the composition for drug delivery, and provides results of measurement of a cell surface expression extent of (A) CD80, (B) MHC II, and (C) MHC I. CD80 is known to act as a costimulatory molecule for T cell activation. The protein was considerably upregulated in the presence of γ-PGA/Ptx/Imq (FIG. 8A). Dose dependent increase in MHC II and MHC I upregulation was also observed for all the samples, and maximized for γ-PGA/Ptx/Imq (FIG. 8B and FIG. 8C). Paclitaxel has been known to enhance maturation of DCs via TLR4 stimulation and imiquimod, a TLR7 agonist, has been known to enhance DC maturation and activation via a MyD88 dependent pathway. Overall, these results demonstrate that the activation and the maturation status of BMDCs were higher in the presence of γ-PGA/Ptx/Imq combination as compared to γ-PGA/Ptx and γ-PGA/Imq only.

### Experimental Example 4

### In-Vivo Anti-Cancer Efficacy

(1) Mice and Cell Lines

Female C57BL/6 (H-2b) mice of 5 to 6 weeks old were purchased from KOATECH (Pyeongtaek, Korea). The mice were maintained under pathogen-free condition. All experiments employing the mice were performed in accordance with the Korean NIH guidelines for care and use of laboratory animals. B16-F10, which is a murine melanoma cell line, was purchased from ATCC. It was allowed to grow in DMEM supplemented with 10% FBS and a 1% antibiotic-antimycotic solution at 37° C. with 5% CO2/95% humidified air.

(2) Evaluation of In-Vivo Antitumor Activity

B16-F10 melanoma cells (1×10⁵) were inoculated into the right flank of 5-6 weeks old C57BL/6 mice. After 1 week of tumor implantation, animals with an average tumor diameter of 4 mm to 6 mm were selected. These mice were divided into five treatment groups (n=7) and numbered. Each drug was administered by intratumoral injection. The drug treatment was continued from day 7 after tumor implantation to day 19 with 3 day interval. Total 4 injections were given (7^{th}, 11^{th}, 15^{th} and 19^{th} days). The tumor diameters were measured till a 23^{rd} day after tumor implantation using a sliding caliper. Tumor volume was calculated using the following formula: tumor volume (mm³)=length×(width)²/2.

On the 23^{rd} day, mice were euthanized, and the tumors were dissected and photographed. For survival study, the treated animals were observed for 41 days. For secondary tumor challenge, tumor-free mice on the 23^{rd} day were rechallenged with 5×10⁴B16-F10 melanoma cells in the opposite flank.

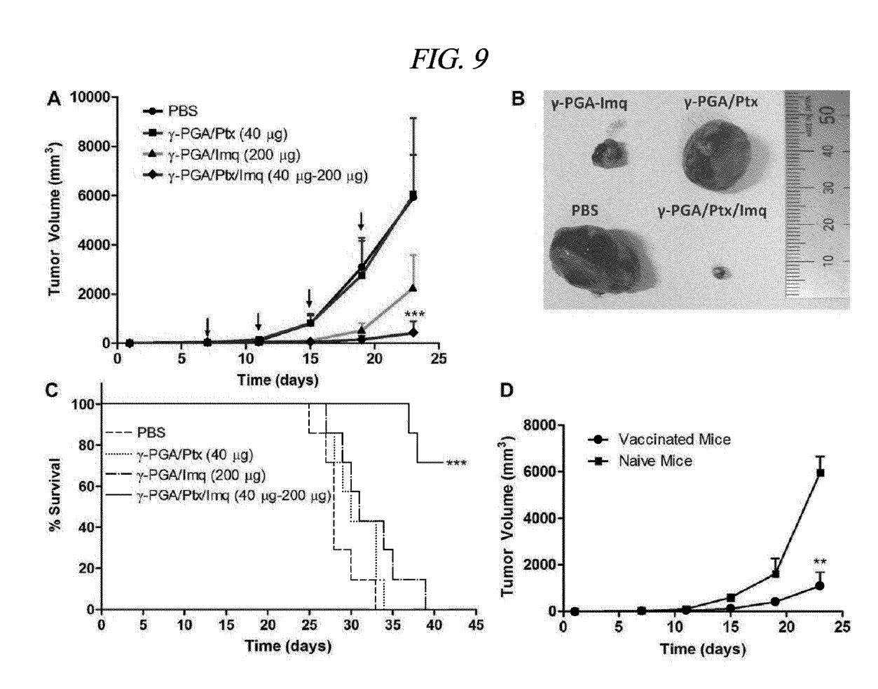

The tumor volume was analyzed every 4 day to test the effectiveness of the complexes. 2 mg/kg (40 μg) of paclitaxel (γ-PGA/Ptx) was insufficient to inhibit the growth of tumor as compared to PBS control. FIG. 9 shows graphs for a tumor volume and survival of a mouse and provides results of measurement of a tumor volume till 23 days after injection of samples. Immuno-modulating agent alone γ-PGA/Imq (200 μg or 10 mg/kg) was able to inhibit the tumor growth to some extent till a 23^{rd} day, but eventually, the tumor size grew larger and led to 0% survival at day 41. Interestingly, treatment with combination γ-PGA/Ptx/Imq including low dose paclitaxel (40 μg) and imiquimod (200 μg) led to drastic inhibition of growth of solid tumor. In FIG. 9A, tumor size data for treatment with each drug had a p value=0.0001, ***p<0.001 by one-way ANOVA and Bonferroni's Multiple Comparison. In FIG. 9C, the survival had a p value=0.0007, ***p<0.001 by Log-rank (ManteleCox) analysis, and in FIG. 9D, the tumor volume analysis till day 23 after tumor rechallenge had a p value=0.0026, **p<0.01 by Unpaired t-test with Welch's correction. 3 out of 7 mice had completely recovered from tumor while the remaining 4 had very small size tumor as shown in FIG. 9B. Moreover, from the survival data, it was found that γ-PGA/ Ptx/Imq led to more than 70% survival of mice till day 41 (FIG. 9C).

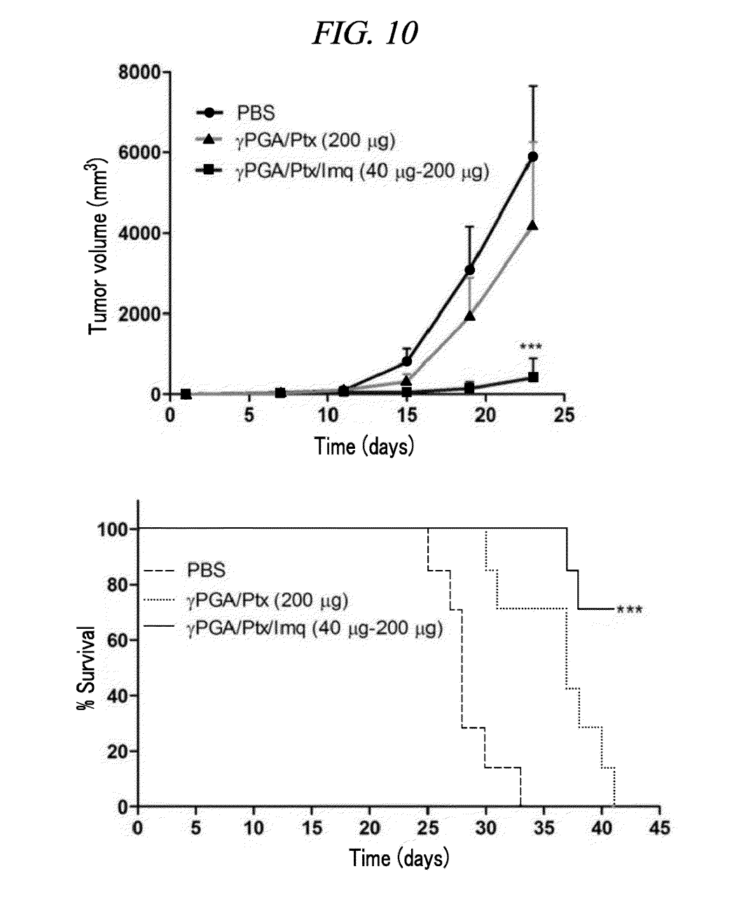

Higher dose of paclitaxel alone in γ-PGA/Ptx (10 mg/kg or 200 μg) was also tested for anti-tumor efficacy. The tumor growth was inhibited to a limited extent and subsequently led to 0% survival at day 41 (FIG. 10). A tumor rechallenge experiment was performed on the γ-PGA/Ptx/Imq treated three mice, which had completely recovered from primary tumor challenge to confirm the presence of anti-tumor memory response. Tumor volume analysis revealed significant inhibition of secondary tumor growth in mice vaccinated with γ-PGA/Ptx/Imq as compared to age matched naïve mice (FIG. 9D). Since the tumor was inoculated in the opposite flank of the mice; this inhibition in tumor growth indicated generation of a systemic tumor specific immune response in the mice after treatment with γ-PGA/Ptx/Imq. These results indicate that the released tumor antigen mediated by low dose of paclitaxel combined with imiquimod mediated dendritic cell activation and maturation is leading to generation of a specific anti-tumor immune response. The systemic memory immune response is crucial in tumor therapy in order to check tumor metastasis and tumor relapse.

### Experimental Example 5

### Evaluation of In-Vivo Immune Status in Tumor Draining Lymph-Node

Mice were challenged with tumor as done in Experimental Example 4 above and treated with 3 intra-tumoral injections. At day 16, the tumor draining lymph node passed through a 70 mm cell strainer in complete RPMI media for isolation of the tumor draining lymph node. The cells were fixed, and then, labeled with fluorescent marker antibodies for specific cell types: FITC anti-mouse CD14 for Macrophage (BD Phar-Mingen, San Diego, Calif., USA), FITC anti-mouse CD11c for Dendritic Cells (BD Phar-Mingen, San Diego, Calif., USA) by incubating at 4° C. for 30 min followed by 2 times washing with PBS. Thereafter, minimum 10⁴ cells were analyzed for fluorescence using MACS (Miltenyl Biotec). Data were further analyzed using MACSQuant Analyzer (Miltenyl Biotec, Germany). Maturation marker (CD40, CD80, CD86 and MHC II) upregulation for CD11c⁺ dendritic cells were further analyzed as done previously.

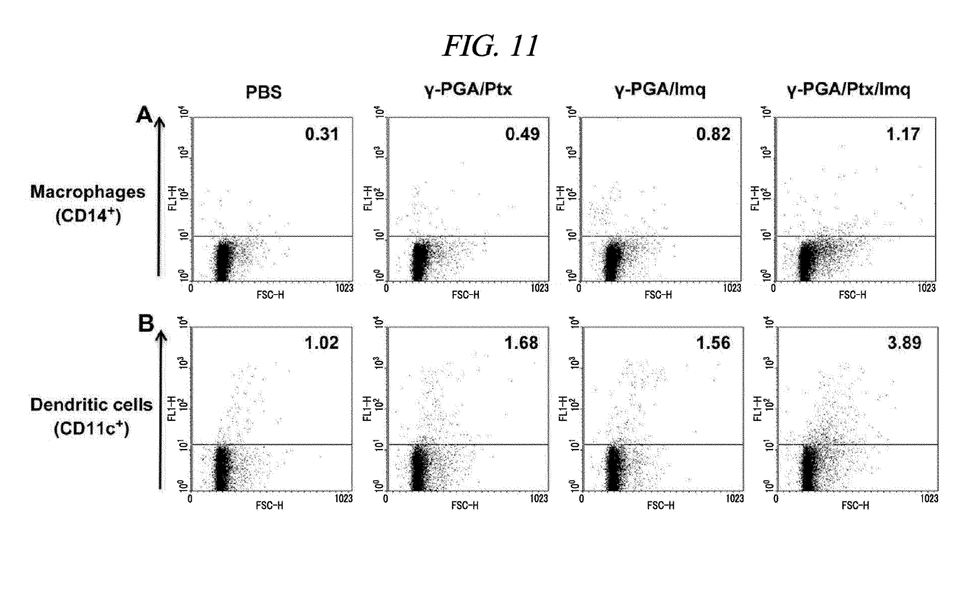

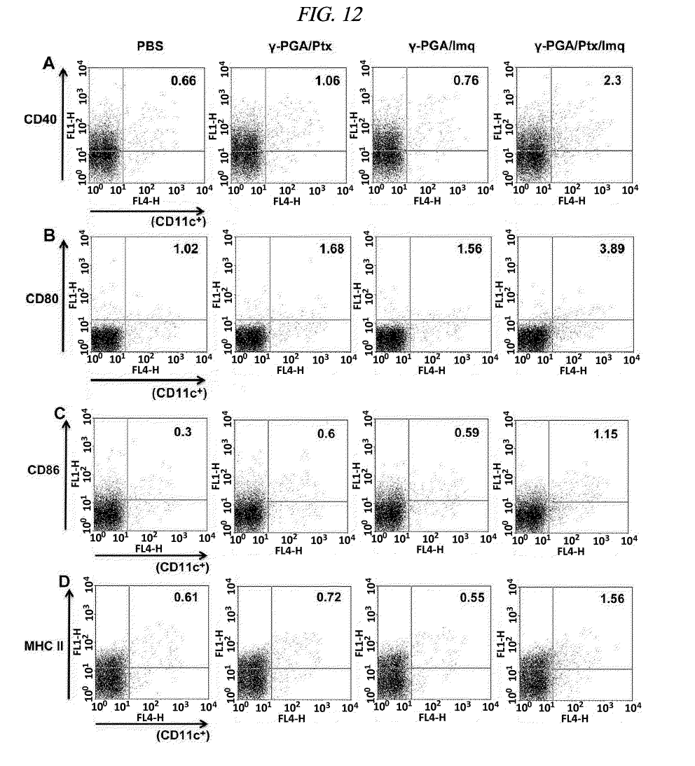

The mice were also tested for immuno-stimulation in-vivo after treatment with all three γ-PGA micro-dispersions. Tumor draining lymph nodes were analyzed for the number of presence of antigen presenting cells (APCs), CD14⁺ macrophages and CD11c+ dendritic cells after treatment with γ-PGA/Ptx, γ-PGA/Imq and γ-PGA/Ptx/Imq. FIG. 11 shows results for immune cell proliferation in tumor draining lymph node on day 16 after administration of the composition for drug delivery, and the mean fluorescence intensity (MFI) indicating a relative number of (A) CD14⁺ macrophages and (B) CD11c⁺ dendritic cells was considerably higher in γ-PGA/Ptx/Imq treated mice as compared to mice treated with PBS (negative control) and individual components γ-PGA/Ptx and γ-PGA/Imq (FIG. 11). FIG. 12 shows results of maturation of CD11c⁺ dendritic cells through CD40, CD80, CD86 and MHC II expression amounts. Upon further analysis of maturation status of CD11c⁺ dendritic cells in the tumor draining lymph node, maturation markers (A) CD40, (B) CD80, (C) CD86 and (D) MHCII were found to be considerably upregulated in γ-PGA/Ptx/Imq treated mice when compared to control (FIG. 12). Both γ-PGA/Ptx and γ-PGA/Imq were able to increase the population of APCs and induce maturation to some extent, because of their TLR4 and TLR7 stimulatory properties respectively. However, this level of stimulation achieved was insufficient for tumor inhibition as observed in a tumor challenge experiment. On the other hand, in combination of the two therapeutic agents in γ-PGA/Ptx/Imq, significant proliferation and maturation of dendritic cells were observed, which are capable of overcoming the immuno-suppressive milieu in tumor and priming T cells in lymph node for generation of a potent anti-tumor immune response.

Effective delivery of a water-insoluble therapeutic agent is one of the major hurdles in their clinical application. Various approaches such as solid dispersions, micro-suspensions and nano-suspensions by wet milling process, melt extrusion or using stabilizers are being explored. The selected anti-cancer drug, paclitaxel, and an immuno-stimulating agent, imiquimod, are water-insoluble in nature. Therefore, the primary aim of an embodiment of the present disclosure was effective administration of the two drugs, for which a water soluble polymer γ-PGA was used, and the drug was allowed to form microdispersion using a co-solvent.

γ-PGA based formulation is particularly beneficial in combination therapy as combinations of various drugs can be readily dispersed in a desired ratio without causing any loss or disturbing the relative dose of individual drugs. γ-PGA has free carboxyl groups, which are expected to have noncovalent interactions such as hydrogen bonding with the amine groups present in drug molecules leading to effective dispersion of drug crystals. The stability of the suspension can also be attributed to the viscosity of the γ-PGA solutions, which prevents precipitation of dispersed micro-crystals. The dispersion was found to be stable without any phase separation up to 6 months.

Especially, one of the most important aims of all anti-cancer immunotherapy is the induction of a potent tumor specific immune response by enabling immune cells to recognize and kill the tumor cells. While cell-based therapy involving dendritic cells, T cells or serial killer cell therapy has been gaining importance and is undergoing various clinical evaluation, these procedures involve multi-step patient procedures. Administration of immune-stimulating agents to produce a robust immune response in-situ can be a convenient approach for anti-cancer immunotherapy. Combination of an immuno-enhancing agent with low amounts of anti-cancer drug in accordance with an embodiment of the present disclosure can facilitate production of a tumor specific antigen.

Cell viability analysis illustrated the effects of individual or combination of both paclitaxel and imiquimod on various tumor cell lines, which were tested in order to verify the versatility of the systems. While paclitaxel containing samples (γ-PGA/Ptx and γ-PGA/Ptx/Imq) had a cytotoxic effect on all the cancer cells lines, a γ-PGA/Imq complex did not show a direct cytotoxic effect on tumor cells. On the other hand, proliferation of BMDCs was dramatically increased in the presence of all the three samples, being highest in case of γ-PGA/Ptx/Imq. Paclitaxel is known to have some dose dependent stimulatory effects on dendritic cells. This is speculated to be a probable reason for enhanced proliferation of BMDCs in the presence of cytotoxic concentrations of paclitaxel both in-vitro and in-vivo. Paclitaxel is also expected to contribute to immuno-stimulation by TLR4 stimulation of DCs and also by inducing tumor apoptosis followed by release of damage associated molecular patterns (DAMPS) such as heat shock proteins (HSPs), high mobility group box-1 (HMBG-1) and calreticulin, which are crucial find me and eat me signals for DCs. These findings when corroborated with the in-vivo anti-tumor efficacy result, clearly elucidate role of individual components. Although γ-PGA/Ptx had direct anti-tumor activity, even higher dose (200 mg) of the drug was not able to completely inhibit tumor growth. The immunostimulatory effect elucidated by γ-PGA/Ptx was insufficient for tumor therapy. Intra-tumoral administration of a γ-PGA/Imq complex, which lacked direct anti-tumor properties, was able to inhibit tumor growth to some extent and probably because imiquimod is also known to impart tumoricidal activity to the DCs enabling them to secrete perforin and granzyme B. With γ-PGA/Imq, even though the tumor growth was delayed, it was not completely inhibited as the tumor size had eventually grown leading to 100% mice death till 41^{st} day. Interestingly, combination of low dose paclitaxel with imiquimod led to drastic inhibition of tumor growth with 70% survival at 41^{st} day. Three out of the seven mice showed complete absence of tumor after 4 doses of γ-PGA/Ptx/Imq. This synergistic effect is attributed to generation of tumor antigens due to presence of low dose paclitaxel, followed by generation of a robust tumor specific immune response due to presence of imiquimod. This immune response is speculated to be majorly mediated by dendritic cells. Usually, tumor infiltrating dendritic cells is inefficient in antigen uptake, antigen presentation and priming of T cells due to various immuno-suppressive mechanisms. With the help of immunostimulatory agents like imiquimod, the dendritic cells can be rendered functional against tumor cells. These mature and activated dendritic cells can recruit and prime other cytotoxic immune cells for tumor depletion. Low dose intra-tumoral administration of paclitaxel reduces systemic side effects associated with chemotherapy, and thus, leads to a potent localized effect. This type of strategy can be called as a bidirectional strategy for treatment of solid tumor, as the anti-cancer drug shall kill the tumor cells from the interior of the solid tumor (inside out), while the diffusing immune-stimulating agents (imiquimod and DAMPs released form dying tumor cells) shall activate immune cells to kill the tumor from the periphery (outside in) (FIG. 1). Thus, this bidirectional chemotherapeutic and immunogenic tumor cell death leads to complete elimination of solid tumor.

Another crucial bottle-neck in successful anti-tumor therapy is metastasis and recurrence, which are associated with all types of cancer leading to generation of secondary tumors and is a major cause of deaths in cancer patients. It poses a challenge for all anticancer modalities as it requires a systemic long lasting a tumor specific immune response for complete eradication of the disease. In the present disclosure, although the drugs were administered intra-tumorally, the response generated was found to be systemic. In 6 weeks after the 4^{th} intra-tumoral injection, significant inhibition in tumor growth was observed in secondary tumor challenge, which was performed on the opposite flank of the mice. This indicates antigen-presenting cells present in the tumor were effectively loaded with tumor antigen and have migrated to tumor draining lymph nodes where priming of T cells takes place. For γ-PGA/Ptx/Imq treated mice, the increase in population of dendritic cells and macrophages in the tumor draining lymph node was confirmed. The maturation status of DCs was also greater in γ-PGA/Ptx/Imq treated mice when compared with control samples. DCs have unique kind of communication with dying tumor cells and depending upon the quantity or nature of cytokines and DAMPs released, the fate of dendritic cells towards immuno-stimulation or tolerance is decided. In case of γ-PGA/Ptx and γ-PGA/Imq samples, although the DCs in the tumor draining lymph nodes are directed towards maturation, the level achieved was not enough for regression of solid tumor. These results clearly signify the central role of APCs (majorly dendritic cells) in production of powerful anti-tumor immune response both locally and systemically. Using such combination therapy seems to be a promising approach for generation of memory response against tumor.

In the Examples and the Experimental Examples of the present disclosure, imiquimod, which is an agonist of a toll-like receptor-7 (TLR-7) as an immuno-stimulating agent, and paclitaxel, which is a low dose chemotherapeutic agent, were synergized to demonstrate tumor therapy along with an anti-tumor memory effect. Both therapeutic agents being water insoluble were dispersed in water with the help of water soluble polymer, poly (γ-glutamic acid) (γ-PGA) using a co-solvent system leading to formation of micro-dispersions of drugs. Paclitaxel and imiquimod formed crystalline microstructures in the size range of 2 μm to 3 μm and were stably dispersed in a γ-PGA matrix for more than 6 months. Paclitaxel and combination of paclitaxel and imiquimod had a significant tumor killing effect in-vitro on various tumor cell lines, while antigen presenting cells (dendritic cells-DCs) treated with the same concentration of imiquimod along with the combination led to enhanced proliferation (250%). In DCs, enhanced secretion of pro-inflammatory and Th1 cytokines was observed in cells co-treated with paclitaxel and imiquimod dispersed in γ-PGA. When administered by intra-tumoral injection in a mouse melanoma tumor model, the treatment with combination exemplified drastic inhibition of tumor growth leading to 70% survival as compared to individual components with 0% survival at 41^{st} day. The anti-tumor response generated was also found to have a systemic memory response since the vaccinated mice significantly deferred secondary tumor development at a distant site 6 weeks after treatment. The relative number and activation status of DCs in-vivo was found to be dramatically increased in case of mice treated with combination. The dramatic inhibition of tumor treated with combination is expected to be mediated by both chemotherapeutic killing of tumor cells followed by uptake of a released antigen by the DCs and due to enhanced proliferation and activation of the DCs.

Standalone chemotherapy or immunotherapy has limitations such as systemic side-effects, insufficient immuno-stimulation, tumor directed tolerance of immune cells, lack of a systemic and tumor specific immune response and tumor recurrence. On the other hand, the present disclosure emphasizes the concept of combination of chemotherapy with immunotherapy for eradication of solid tumors using water insoluble therapeutic agents. While most of these agents are water-insoluble in nature, using poly (γ-glutamic acid) for formulation of micro-dispersions of therapeutic moiety seems to be a simple and practicable approach. The low dose of anti-cancer drug paclitaxel is expected to have minimal side-effects but capable of generating a sufficient amount of tumor antigens and danger signals for generation of a tumor specific immune response. In a mouse model, intratumoral administration of imiquimod combined with paclitaxel dispersed in a γ-PGA matrix has led to powerful immunogenic tumor cell death followed by inhibition of secondary tumors indicating presence of a memory effect. This combination has shown potential for both therapeutic as well as prophylactic anti-tumor therapy. The versatility of the approach can be further elucidated by exploring various combinations of anti-cancer drugs and immunestimulating agents.

The above description of the embodiments is provided for the purpose of illustration, and it would be understood by those skilled in the art that various changes and modifications may be made without changing technical conception and essential features of the embodiments. Thus, it is clear that the above-described embodiments are illustrative in all aspects and do not limit the present disclosure. For example, each component described to be of a single type can be implemented in a distributed manner. Likewise, components described to be distributed can be implemented in a combined manner.

The scope of the inventive concept is defined by the following claims and their equivalents rather than by the detailed description of the embodiments. It shall be understood that all modifications and embodiments conceived from the meaning and scope of the claims and their equivalents are included in the scope of the inventive concept.

We claim:

1. A composition for drug delivery, comprising
a biocompatible anionic polymer;
an agonist of an immunostimulant; and
an active pharmaceutical ingredient.

2. The composition for drug delivery of claim 1,
wherein the biocompatible anionic polymer includes a member selected from the group consisting of carboxyl group, hydroxyl group, sulfonic group, sulfate group, and combinations thereof.

3. The composition for drug delivery of claim 1,
wherein the biocompatible anionic polymer includes a member selected from the group consisting of a poly-gamma-glutamic acid, a hyaluronic acid, a cellulose, a polyacrylic acid, a polyamino acid, a polysaccharide, derivatives thereof, and combinations thereof.

4. The composition for drug delivery of claim 1,
wherein the immunostimulant includes a toll-like receptor, a NOD-like receptor, or a cytokine.

5. The composition for drug delivery of claim 1,
wherein the agonist of the immunostimulant includes poly(I:CU), CpG, imiquimod, resiquimod, dSLIM, MPLA, flagellin, a plasmid DNA double-strand DNA, a single-strand DNA, a saponin, or an interleukin cytokine.

6. The composition for drug delivery of claim 1,
wherein the active pharmaceutical ingredient includes an anticancer agent.

7. The composition for drug delivery of claim 6,
wherein the anticancer agent includes a member selected from the group consisting of paclitaxel, docetaxel, doxorubicin, adriamycin, cis-platin, mitomycin-C, daunomycin, 5-fluorouracil, griseofulvin, digoxin, dipyridamol, spironolactone, cyclosporine, amphotericin B, etoposide, 6-mercaptopurine, dexamethasone, perphenazine, 20-S-camptothecin, 9-nitro-camptothecin, 9-amino-camptothecin, 10,11-methylenedioxy-camptothecin, taxol, taxol-A, mitotane, methotrexate, lomustine, interferon, rouracil, and etoposide.

8. The composition for drug delivery of claim 1,
wherein the biocompatible anionic polymer, the agonist of the immunostimulant and the active pharmaceutical ingredient are linked to each other via an intermolecular non-covalent bond.

9. A method of producing a composition for drug delivery of claim 1, comprising:
(a) adding a biocompatible anionic polymer into an agonist of the immunostimulant dispersed in an organic solvent under ultrasonication to prepare a dispersion of the biocompatible anionic polymer-the agonist of the immunostimulant, and then lyophilizing the dispersion to remove the organic solvent so as to obtain a powder of the agonist of the immunostimulant dispersed in the biocompatible anionic polymer;
(b) adding a biocompatible anionic polymer into an active pharmaceutical ingredient dispersed in an organic solvent under ultrasonication to prepare a dispersion of the biocompatible anionic polymer-the active pharmaceutical ingredient, and then lyophilizing the dispersion to remove the organic solvent so as to obtain a powder of the active pharmaceutical ingredient dispersed in the biocompatible anionic polymer; and
(c) redispersing the powders obtained from the steps (a) and (b) in distilled water.

10. The method of claim 9,
wherein the biocompatible anionic polymer includes a polymer including a member selected from the group consisting of carboxyl group, hydroxyl group, sulfonic group, sulfate group, and combinations thereof.

11. The method of claim 9,
wherein the biocompatible anionic polymer includes a member selected from the group consisting of a poly-gamma-glutamic acid, a hyaluronic acid, a cellulose, a polyacrylic acid, a polyamino acid, a polysaccharide, derivatives thereof, and combinations thereof.

12. The method of claim 9,
wherein the immunostimulant includes a toll-like receptor, a NOD-like receptor, or a cytokine.

13. The method of claim 9,
wherein the agonist of the immunostimulant includes poly(I:CU), CpG, imiquimod , resiquimod, dSLIM, MPLA, flagellin, a plasmid DNA double-strand DNA, a single-strand DNA, a saponin, or an interleukin cytokine.

14. The method of claim 9,
wherein the active pharmaceutical ingredient includes an anticancer agent.

15. The method of claim 14,
wherein the anticancer agent includes a member selected from the group consisting of paclitaxel, docetaxel, doxorubicin, adriamycin, cis-platin, mitomycin-C, daunomycin, 5-fluorouracil, griseofulvin, digoxin, dipyridamol, spironolactone, cyclosporine, amphotericin B , etoposide, 6-mercaptopurine, dexamethasone, perphenazine, 20-S-camptothecin, 9-nitro-camptothecin, 9-amino-camptothecin, 10,11-methylenedioxy-camptothecin, taxol, taxol-A, mitotane, methotrexate, lomustine, interferon, rouracil, and etoposide.

16. The method of claim 9,
wherein the agonist of the immunostimulant dispersed in the biocompatible anionic polymer and the active pharmaceutical ingredient dispersed in the biocompatible anionic polymer are linked to each other via an intermolecular non-covalent bond.

17. The method of claim 9,
wherein the biocompatible anionic polymer and the agonist of the immunostimulant in the agonist of the immunostimulant dispersed in the biocompatible anionic polymer are included in a weight ratio of 1:0.01 to 300;
wherein the biocompatible anionic polymer and the active pharmaceutical ingredient in the active pharmaceutical ingredient dispersed in the biocompatible anionic polymer are included in a weight ratio of 1:0.01 to 300; and
wherein the agonist of the immunostimulant dispersed in the biocompatible anionic polymer and the active pharmaceutical ingredient dispersed in the biocompatible anionic polymer are included in a weight ratio of 1:1 to 5.

18. A composition for anticancer therapy, comprising a composition for drug delivery of claim 1 as an active ingredient.
